(19) United States
(12) Patent Application Publication
(10) Pub. No.: US 2017/0232276 A1
(43) Pub. Date: Aug. 17, 2017

(54) TREATMENT OF FIBROSIS USING DEEP TISSUE HEATING AND STEM CELL THERAPY

(71) Applicant: PrimeGen Biotech, LLC, Santa Ana, CA (US)

(72) Inventor: Thomas C.K. Yuen, Santa Ana, CA (US)

(21) Appl. No.: 15/514,904

(22) PCT Filed: Sep. 30, 2015
(86) PCT No.: PCT/US15/53131

§ 371 Date: Mar. 28, 2017

Related U.S. Application Data
(60) Provisional application No. 62/057,933, filed on Sep. 30, 2014.

(51) Int. Cl. A61N 7/02; A61N 5/06

(52) U.S. Cl. CPC A61N 7/02; A61N 5/0625; A61N 2005/067

(57) ABSTRACT

Provided are methods for treating fibrosis of tissues or organs in a subject in need thereof comprising the steps of administering a therapeutically effective amount of energy from an energy source to the targeted tissue or organ; and administering a therapeutically effective amount of stem cells to the tissue or organ. Further provided are methods for monitoring fibrosis in the treated tissue or organ after administration of the energy.

### FIELD OF THE INVENTION

The invention relates to methods and compositions for treating tissue and organ fibrosis using a combination of deep tissue heating induced by the application of energy followed by the administration of stem cells.

### BACKGROUND

Fibrosis is a pathogenic condition characterized by an excessive accumulation and deposition of extracellular matrix (ECM), immunocomponents, connective, or scar tissue in and around organs, joints, and other bodily tissues. Fibrosis can lead to scarring, inflammation, or damage and, in some cases, the ultimate failure of the tissue or organ. Fibrosis can affect any bodily tissue or organ, including the liver, kidneys, lungs, heart, intestine, skin, joints, and uterus. Renal fibrosis, for example, is associated with chronic kidney disease (CKD). The resulting kidney failure can only be treated using dialysis or kidney transplantation. Thus, there is a need in the art for methods that can arrest or reverse tissue and organ fibrosis before irreparable damage is done.

### SUMMARY

The present disclosure is based, at least in part, on the benefits of treating fibrosis using a combination of deep tissue energy application to arrest and/or reduce fibrosis and administration of stem cells to repair and/or regenerate the damaged tissue or organ.

In one embodiment, provided is a method for treating fibrosis of a tissue or an organ in a subject in need thereof comprising the steps of administering a therapeutically effective amount of energy from an energy source to the affected tissue or organ; followed by administering a therapeutically effective amount of stem cells to the subject in and/or around the targeted tissue or organ. In some embodiments, the energy induces heating in the target organ. In other embodiments, the claimed method may treat fibrotic conditions including, but not limited to, treating thickening and scarring of connective tissue resulting from injury, trauma, non-trauma, surgery, hereditary disease, or other chronic or non-chronic conditions.

In still other embodiments, the method may treat conditions such as, but not limited to, adhesive capsulitis, arterial fibrosis, arthrofibrosis, Crohn's disease, cirrhosis, cystic fibrosis, Dupuytren's contracture, endomyocardial fibrosis, fibroleiomyoma, fibromyoma, idiopathic pulmonary fibrosis, keloid, mediastinal fibrosis, myelofibrosis, nephrogenic systemic fibrosis, old myocardial infarction, myoma, Peyronie's disease, progressive massive fibrosis, pulmonary fibrosis, retroperitoneal fibrosis, scleroderma/systemic sclerosis, uterine fibroids, uterine leiomyoma, and other conditions relating to excessive connective tissue.

In a further embodiment, the method comprises detecting the level of fibrosis in the tissue or in the organ of the subject after administration of the energy; and administering the therapeutically effective amount of stem cells to the subject only if fibrosis in the tissue or organ of the subject is reduced after administration of the energy. In one embodiment, the level of fibrosis is detected using a non-invasive imaging method. In a further embodiment, the level of fibrosis is detected using an imaging method selected from computed tomography (CT), magnetic resonance imaging (MRI), ultrasound, and optical tomography. In another embodiment, detecting the level of fibrosis in the tissue or organ comprises obtaining a biological sample from the subject; measuring the amount of at least one marker of fibrosis in the biological sample; and comparing the amount of at least one marker of fibrosis to a reference value. In one embodiment, the reference value is the amount of the at least one marker of fibrosis in a biological sample (e.g., blood, plasma, serum, urine, or tissue, including a tissue sample from the organ) obtained from the subject prior to the administration of the energy, and wherein a reduction in the amount of the at least one marker of fibrosis as compared to the reference value indicates a reduction of fibrosis in the organ of the subject. In another embodiment, the reference value is the amount of the at least one marker of fibrosis in a biological sample from a subject or subjects known to have fibrosis, and wherein a reduction in the amount of the at least one marker of fibrosis as compared to the reference value indicates a reduction of fibrosis in the organ of the subject.

Markers of fibrosis may include, but are not limited to, urea, creatinine, and blood urea nitrogen, as well as fibroblast-specific protein 1 (FSP-1), α-smooth muscle actin (α-SMA), interleukin 6 (IL-6), monocyte chemotactic protein-1 (MCP-1), transforming growth factor β1 (TGF-β1), and Smad3.

In some embodiments, the steps of energy administration and/or detection of at least one marker of fibrosis are repeated at least one or more times. In another embodiment, the step of detection of at least one marker of fibrosis is performed once weekly.

In one embodiment, the energy source may be a low-level laser (e.g., an A1GaAs laser). In another embodiment, the energy source may be a pulsed laser, calibrated laser, adjustable laser, or continuous wave laser. In a further embodiment, the therapeutically effective amount of energy may comprise a wavelength of about 780 nm, a power output of about 3 mW, and a beam area of about 4 mm², and wherein the energy is applied for about 30 seconds. In a further embodiment, the energy may be delivered at a plurality of points on the surface of the organ, and wherein the energy is applied for about 30 seconds at each point. In a further embodiment, the delivered energy density may be about 22.5 J/cm². In another embodiment, the energy source is ultrasound (e.g., high-intensity focused ultrasound).

In one embodiment, the fibrosis-affected area may be an organ, joint, or other areas of the body with fibroids or excessive connective or scar tissue. In another embodiment, the treated organ may be, but is not limited to, the kidney, liver, heart, lung, skin, intestine, or uterus.

In a further embodiment, the stem cells may be bone marrow-derived stem cells, such as mesenchymal stem cells (MSC). In other embodiments, the stem cell may be adult derived stem cells. In another embodiment, the stem cells may be adipose-derived stem cells (ADSC), for example, adipose-derived side population stem cells (ADSC-SP). In some embodiments, the stem cells may be germline stem cells. Germline stem cells generally will form into gametes and have high conservation, regulation, and quality of DNA. Germline stem cells may retain a multipotent ability and may generate all three germ layers through laboratory experimentation. In a further embodiment, the stem cells may be isolated from a tissue selected from the group consisting of bone marrow, skeletal muscle, umbilical cord, synovium, blood, dental pulp, amniotic fluid, fetal blood, lung, gonadal tissue, and liver. In another embodiment, the stem cells may be induced pluripotent stem cells (iPSC). In a further embodiment, the stem cells may be autologous to the subject.

In another embodiment, the stem cells may be administered via a route selected from the group consisting of parenteral, intravenous, and subcutaneous. In a further embodiment, the stem cells may be administered at the site of the affected bodily tissue or organ.

In another embodiment, the method further comprises the step of administering to the subject a therapeutically effective amount of at least one agent for treating fibrosis (e.g., an anti-inflammatory or anti-fibrotic agent and/or at least one agent for preventing graft rejection).

In another embodiment, the subject may be a mammal or a bird. In another embodiment, the subject may include a human, a non-human primate, or a livestock, veterinary, zoo, or companion animal. In a further embodiment, the subject may be selected from the group consisting of: a mouse, rat, rabbit, cat, dog, chimpanzee, monkey, cow, bull, horse, goat, sheep, pig, chicken, and human.

### DETAILED DESCRIPTION OF THE INVENTION

The present disclosure is based, at least in part, on benefits of treating fibrosis using a combination of deep tissue energy application to arrest and/or reduce fibrosis and administration of stem cells to repair and/or regenerate the affected bodily tissue or organ. Without intending to be bound by theory, the methods disclosed herein address a two-fold need in a method to treat fibrosis. First, there is a need to arrest and/or reverse the fibrotic process; and second, there is a need to repair damage already done to the tissue. Without a repair step, a method for stopping fibrosis may be inadequate because of irreparable damage already done to the tissue or organ. Without a step that stops fibrosis, a repair step on its own may be inadequate because the fibrotic process will continue to damage the tissue or organ. Additionally, even if the fibrotic process is prevented from progressing, the existing fibrosis may block repair of the tissue or organ, for example, by blocking entry and dispersal of the stem cells into the damaged portions of the tissue or organ, and by choking off existing blood vessels and/or blocking new blood vessel growth (e.g., angiogenic sprouting). Without perfusion by an adequate blood supply, repair of a damaged tissue or organ may be prevented. Accordingly, the methods disclosed herein may act not only to prevent further fibrosis from occurring, but to reverse existing fibrosis and thus allow penetration and dispersal of the administered stem cells throughout the tissue and/or organ, which then may initiate angiogenesis and repair.

Accordingly, in one embodiment, provided is a method for treating fibrosis of a tissue or organ in a subject in need thereof comprising the steps of administering a therapeutically effective amount of energy from an energy source to the affected tissue or organ; followed by administering a therapeutically effective amount of stem cells to the subject.

As used herein the term “fibrosis” may refer to the pathogenic condition characterized by an excessive accumulation and deposition of extracellular matrix (ECM) and immunocomponents, connective, or scar tissue in and around organs, joints and other bodily tissues. The term “fibrosis” also may refer to the process (also referred to as the “fibrotic process”) by which the condition of fibrosis occurs. Thus, the phrase “reduce fibrosis” (and the like) may refer either to reduction in the state (e.g., the amount) of fibrosis in an organ, or a reduction in the process by which fibrosis occurs.

The energy source may be any energy source capable of delivering energy to the affected tissue or organ inside a subject's body, such that deep heating is induced in the tissue or organ. The type and amount of energy used should be sufficient to arrest, inhibit, or reduce fibrosis in the tissue or organ, but should preferably not harm or damage the targeted tissue or organ or any other tissues or organs in the subject. For example, in one embodiment, pulse modulation of a laser may be used to balance the need for power high enough to penetrate sufficiently deep within the body to reach the target organ (e.g., at least about 1, 2, 3, 4, 5, 6, 7 8, 9, 10 or more cm from the surface of the body) without damaging other tissues or organs (e.g., skin, blood vessels, nerves, fascia, muscle, adipose tissue, etc.) positioned between the target organ and the surface of the body.

In one embodiment, the energy source may be a low-level laser (e.g., an A1GaAs laser). Therapy comprising application of energy using a low-level laser may be referred to interchangeably as “low-level light therapy” or “low-level laser therapy” (“LLLT”), or as “low-intensity light therapy” or “low intensity laser therapy” (“LILT”). For example, the energy source may be a pulsed laser, such as the pulsed tunable laser (e.g., a Kerr-lens self-mode-locked (KLM) titanium sapphire (Ti:S) laser) described in US Patent Publication No. 20100/265493, incorporated herein by reference for all it discloses regarding pulsed tunable lasers. In another embodiment, the energy source may be a calibrated laser. Methods and equations for calibrating various parameters of a pulsed laser (e.g., pulse rate, pulse energy, etc.) may be similar to those described for use in eye surgery, adapted for use in treating organs as described herein. Such methods are described, for example, in US Patent Publication Nos. 2011/0267446, 2007/0213697, and 2011/0028956, all of which are incorporated herein by reference for all they disclose regarding calibrated lasers. Low-level lasers for use in delivering energy to an organ are further described in, for example, US Patent Publication Nos. 2003/0144712, 2003/0212442, 2004/0220513, 2004/0260367, 2004/0153130, and 2010/0016783, all of which are incorporated herein by reference for all they disclose regarding low-level lasers. Additional description of lasers, including continuous wave lasers, useful in the methods described herein, may be found, for example, in US Patent Publication Nos. 2012/0302879, 2013/0211388, 2013/0184693, and 2013/0102880, all of which are incorporated herein by reference for all they disclose regarding lasers, including continuous wave lasers.

In a further embodiment, the therapeutically effective amount of energy may comprise a wavelength in the infrared (IR) or near-infrared (NIR) spectrum, for example, having a wavelength of about 600-1100 nm, including ranges of about 600-700 nm, 650-750 nm, 700-800 nm, 750-850 nm, 800-900 nm, 850-950 nm, 900-1000 nm, 950-1050 nm, 1000-1100 nm, 650-1050 nm, 700-1000 nm, or 750-950 nm, or including about 600 nm, 620 nm, 625 nm, 650 nm, 675 nm, 700 nm, 725 nm, 750 nm, 775 nm, 780 nm, 785 nm, 790 nm, 800 nm, 810 nm, 820 nm, 825 nm, 850 nm, 875 nm, 900 nm, 925 nm, 950 nm, 975 nm, 1000 nm, 1025 nm, 1050 nm, 1075 nm, or 1100 nm. In other embodiments, the wavelength may be about 780 nm.

In another embodiment, the laser may comprise a power output of about 1-500 mW, including ranges of about 1-5 mW, 1-10 mW, 1-15 mW, 1-20 mW, 1-25 mW, 1-30 mW, 1-35 mW, 1-40 mW, 1-45 mW, 1-50 mW, 1-75 mW, 1-100 mW, 5-10 mW, 10-15 mW, 15-20 mW, 20-25 mW, 25-30 mW, 30-35 mW, 35-40 mW, 40-45 mW, 45-50 mW, 5-45 mW, 10-40 mW, 15-35 mW, 20-30 mW, 50-150 mW, 100-200 mW, 150-250 mW, 200-300 mW, 250-350 mW, 300-400 mW, 350-450 mW, 400-500 mW, 50-450 mW, 100-300 mW, or including about 3 mW, about 5 mW, about 10 mW, about 15 mW, about 20 mW, about 25 mW, about 30 mW, about 35 mW, about 40 mW, about 45 mW, about 50 mW, about 60 mW, about 70 mW, about 80 mW, about 90 mW, about 100 mW, about 150 mW, about 200 mW, about 250 mW, about 300 mW, about 350 mW, about 400 mW, about 450 mW, or about 500 mW. In other embodiments, the power output may be about 3 mW.

In another embodiment, the laser may emit a beam area of about 4 mm² or about 0.1256 cm². In another embodiment, the laser may emit a beam area of about 0.1-10 mm², including ranges of about 0.5-9.5 mm², 1-9 mm², 2-8 mm², 3-7 mm², 4-6 mm², 1-7 mm², 2-6 mm², 3-5 mm², 0.1-1 mm², 1-2 mm², 2-3 mm², 3-4 mm², 4-5 mm², 5-6 mm², 6-7 mm², 8-9 mm², 9-10 mm², or about 0.1 mm², 0.5 mm², 1 mm², 1.5 mm², 2 mm², 2.5 mm², 3 mm², 3.5 mm², 4 mm², 4.5 mm², 5 mm², 5.5 mm², 6 mm², 6.5 mm², 7 mm², 7.5 mm², 8 mm², 8.5 mm², 9 mm², 10 mm² or more. In other embodiments, the beam area may be about 4 mm².

In another embodiment, the energy may be applied for about 1-120 seconds, including ranges of about 1-5, 1-10, 1-20, 1-25, 1-30, 1-35, 1-40, 1-45, 1-50, 1-60, 1-70, 1-80, 1-90, 1-100, 1-110, 5-10, 10-15, 15-20, 20-25, 25-30, 35-40, 45-50, 50-55, 55-60, 60-70, 70-80, 80-90, 90-100, 100-110, or 110-120 seconds, or about 1, 2, 3, 4, 5, 10, 15, 20, 25, 30, 35, 40, 45, 50, 55, 60, 65, 70, 75, 80, 85, 90, 95, 100, 105, 110, 115, or 120 seconds. In other embodiments, the energy may be applied for about 30 seconds.

In another embodiment, the laser may have a power density (irradiance) of between about 1 mW-5 W/cm², including ranges of about 1-5, 1-10, 1-15, 1-20, 1-25, 1-30, 1-35, 1-40, 1-45, 1-50, 1-75, 1-100, 1-150, 1-200, 1-250, 1-300, 1-350, 1-400, 1-450, 1-500, 1-600, 1-700, 1-800, 1-900, 1-1000, 1-2000, 1-3000, 1-4000, 5-10, 10-20, 20-30, 30-40, 40-50, 50-100, 100-200, 200-300, 300-400, 400-500, 500-1000, 1000-2000, 2000-3000, 3000-4000, or 4000-5000, or about 1, 5, 10, 15, 20, 25, 30, 35, 40, 45, 50, 75, 100, 150, 200, 250, 300, 350, 400, 450, 500, 600, 700, 800, 900, 1000, 2000, 3000, 4000, or 5000 mW/cm².

In a further embodiment, the energy may be delivered at a plurality of points on the surface of the organ, and wherein the energy may be applied for about 30 seconds at each point. In a further embodiment, the delivered energy density may be about 1-30 J/cm², including ranges of about 1-5 J/cm², 1-10 J/cm², 1-15 J/cm², 1-20 J/cm², 1-25 J/cm², 5-10 J/cm², 10-15 J/cm², 15-20 J/cm², 20-25 J/cm², 25-30 J/cm², 4-25 J/cm², 5-25 J/cm², 10-20 J/cm², or including about 4 J/cm², about 5 J/cm², about 7.5 J/cm², about 10 J/cm², about 12.5 J/cm², about 15 J/cm², about 17.5 J/cm², about 20 J/cm², about 22.5 J/cm². In other embodiments, the delivered energy density may be about 22.5 J/cm².

In another embodiment, the pulse repetition rate (PRR) may be from about 1 to about 5000 pulses per second (pps), including ranges of about 1-5, 1-10, 1-20, 1-30, 1-40, 1-50, 1-75, 1-100, 1-150, 1-200, 1-250, 1-300, 1-350, 1-400, 1-450, 1-500, 1-600, 1-700, 1-800, 1-900, 1-1000, 1-2000, 1-3000, 1-4000, 5-10, 10-20, 20-30, 30-40, 40-50, 50-100, 100-200, 200-300, 300-400, 400-500, 500-1000, 1000-2000, 2000-3000, 3000-4000, or 4000-5000, or about 1, 2, 2.5, 3, 4, 5, 6, 7, 8, 9, 10, 15, 16, 20, 25, 30, 35, 40, 45, 50, 60, 70, 80, 90, 100, 125, 150, 160, 175, 200, 250, 292, 300, 350, 400, 450, 500, 600, 700, 800, 900, 1000, 2000, 3000, 4000, or 5000 pps. In other embodiments, the PRR may be about 5000 pps.

In another embodiment, the pulse repetition frequency may be about 1-100 mHz, including ranges of about 1-25, 1-50, 1-70, 1-80, 1-90, 50-100, 60-100, 70-100, 80-100, 90-100, 70-90, mHz, or including 1, 5, 10, 15, 20, 25, 30, 35, 40, 45, 50, 55, 60, 65, 70, 75, 80, 85, 90, 95, or 100 mHz.

In another embodiment, the energy source may be ultrasound (e.g., high-intensity focused ultrasound. In other embodiments, the energy source may comprise incandescent light, light emitting diodes, and/or super luminous diodes.

In another embodiment, the energy applied to the affected tissue or organ may be mechanical in nature, e.g., mechanical vibration similar to that used in the art to eliminate kidney, bladder, or gall stones. Such mechanical vibration may be induced using ultrasound (e.g., high-frequency ultrasound) or electromechanical vibration. In another embodiment, mechanical vibration may be combined with another form of energy as described herein. For example, in one embodiment, mechanical vibration may be combined with the use of low-level laser therapy.

Additional forms of energy capable of inducing deep-tissue heating are known to those of skill in the art.

Further embodiments of the methods described herein may combine multiple different forms of energy application, either simultaneously or sequentially.

In some embodiments, the subject may be monitored after treatment with the energy for changes in markers of fibrosis before administering stem cells. Because some methods of administering stem cells may be at least moderately invasive, and because some methods of administering stem cells may be associated with undesirable side effects (e.g., unwanted immune responses), it may be desirable to administer the stem cells only after it is determined that fibrosis of the target tissue or organ has been arrested and/or reduced. Accordingly, provided herein may be a monitoring step useful for determining whether fibrosis in the affected tissue or organ has been arrested and/or reduced by the application of energy. Thus, some embodiments of the methods herein may comprise detecting the level of fibrosis in the organ of the subject; and administering the therapeutically effective amount of stem cells to the subject only if fibrosis in the affected tissue or organ of the subject is reduced after administration of the energy.

Detection of the level of fibrosis in the affected tissue or organ may comprise obtaining a biological sample from the subject; measuring the amount of at least one marker of fibrosis in the biological sample; and comparing the amount of the at least one marker of fibrosis to a reference value. In one embodiment, the reference value may be the amount of the at least one marker of fibrosis in a biological sample (e.g., blood, plasma, serum, urine, or tissue, including a tissue sample from the affected tissue or organ) obtained from the subject prior to the administration of the energy, and wherein a reduction in the amount of the at least one marker of fibrosis as compared to the reference value indicates a reduction of fibrosis in the affected tissue or organ of the subject. In another embodiment, the reference value is the amount of the at least one marker of fibrosis in a biological sample from a subject or subjects known to have fibrosis, and wherein a reduction in the amount of the at least one marker of fibrosis as compared to the reference value indicates a reduction of fibrosis in the affected tissue or organ of the subject.

Markers of fibrosis include, but are not limited to, urea, creatinine, and blood urea nitrogen, as well as protein or nucleic acid biomarkers such as fibroblast-specific protein 1 (FSP-1), α-smooth muscle actin (α-SMA), interleukin 6 (IL-6), monocyte chemotactic protein-1 (MCP-1), transforming growth factor β1 (TGF-β1), or Smad3.

In some embodiments, detection of the level of fibrosis may comprise the use of non-invasive imaging, including functional and/or molecular imaging. For example, determination of fibrosis via functional imaging of blood vessels in organs may be performed using computed tomography (CT), e.g., contrast-enhanced micro-CT. The level of fibrosis may also be determined by imaging ECM deposition using magnetic resonance imaging (MRI) (e.g., T1-weighted MRI) with an elastin-specific magnetic resonance imaging agent (ESMA). In some embodiments, detection of the level of fibrosis may comprise the use of laser imaging techniques using, for example, the low-level, pulsed, or continuous wave lasers described herein. Systems for laser imaging are described, for example, in US Patent Publication Nos. 2010/0265493, 2012/0302879, 200/90292202, 2013/0102880, 2005/0090750, 2005/0171414, and 2007/0093702, all of which are incorporated herein by reference for all they disclose regarding laser imaging. In other embodiments, detection of the level of fibrosis may be determined using ultrasound imaging and/or optical tomography.

In some embodiments, the steps of energy administration and/or monitoring the level of fibrosis may be repeated at least one or more times. For example, the administration of energy may be given in more than one dose. In some embodiments, the energy may be administered in a plurality of doses per day, per week, per month, or per year, and the frequency of doses may be hourly, daily, weekly, or monthly. In another embodiment, the monitoring step may be performed a plurality of times per day, per week, per month, or per year, and the frequency of monitoring may be hourly, daily, weekly, or monthly. In one embodiment, the monitoring may be performed once per week. In another embodiment, the energy is administered one time, and the monitoring step is performed a plurality of times until a reduction in fibrosis is observed. In another embodiment, the monitoring step may be performed once after each dose of energy is administered. For example, in one embodiment, the energy may be administered once per week, followed by a monitoring step, until a reduction in fibrosis is observed.

Stem cells for use in the methods disclosed herein may be any known type of stem cells capable of repairing or regenerating an organ.

In one embodiment, the stem cells may be bone marrow-derived stem cells, such as mesenchymal stem cells (MSC). Mesenchymal stem cells can be isolated from a variety of tissues, including, but not limited to, skeletal muscle, adipose tissue, umbilical cord, synovium, the circulatory system (e.g., blood), dental pulp, amniotic fluid, fetal blood, lung, liver, gonadal tissue, and bone marrow. Mesenchymal stem cells can be further identified using the minimal criteria established by the Mesenchymal and Tissue Stem Cell Committee of the International Society for Cellular Therapy, which is hereby incorporated by reference in its entirety. First, MSC must be plastic-adherent when maintained in standard culture conditions. Second, MSC must express CD105, CD73 and CD90, and lack expression of CD45, CD34, CD14 or CD11 b, CD79a or CD19 and HLA-DR surface molecules. Third, MSC must differentiate to osteoblasts, adipocytes and chondroblasts in vitro (Dominici et al. “Minimal criteria for defining multipotent mesenchymal stromal cells. The International Society for Cellular Therapy position statement.” (2006) Cytotherapy 8(4):315-317), which is hereby incorporated by reference in its entirety.

In another embodiment, the stem cells may be adult stem cells, such as adipose-derived stem cells (ADSC), for example, adipose-derived side population stem cells (ADSC-SP). ADSC-SP stem cells are described, for example, in US Patent Publication No. 2006/0147430, incorporated herein by reference for all it contains regarding ADSC and ADSC-SP cells.

In another embodiment, the stem cells are induced pluripotent stem cells (iPS or iPSC). iPS cells are described, for example, in US Patent Publication No. 2009/0068742 and US Patent Publication No. 2012/0282229, incorporated herein by reference for all they contain regarding iPS cells.

In other embodiments, the stem cells may be germline stem cells. Germline stem cells are characterized by having high conservation, regulation and quality of genetic material. Germline stem cells are generally the source of gametes within organisms and are able to self-renew to maintain germline stem cell levels or allow for differentiation. Germline stem cells are described, for example, in US Patent Publication No. 2010/0285577, hereby incorporated by reference herein for all it contains regarding germline stem cells.

In a further embodiment, the stem cells may be autologous to the subject. If available, autologous stem cells may be beneficial to the subject because they will reduce or eliminate the potential for adverse immune responses, e.g., rejection of the stem cells or graft-versus-host disease. Autologous stem cells may be, e.g., stem cells isolated directly from the subject (e.g., mesenchymal stem cells), or iPS cells produced from non-stem cells from the subject (e.g., iPS cells produced using the methods described in US Patent Publication No. 2009/0068742 and US Patent Publication No. 2012/0282229).

In another embodiment, in cases where autologous stem cells are not available or not indicated for a particular subject, allogeneic stem cells may be used. Allogeneic stem cells should be matched as closely as possible to the subject (e.g., via HLA genotype) in order to reduce the likelihood of rejection or graft-versus-host disease. In one embodiment, the stem cell donor is a first-degree-relative (e.g., parent, sibling, or child) of the subject, which increases the likelihood of finding a closely-matched donor. In another embodiment, the stem cell donor may be an extended relative of the subject. In another embodiment, the stem cell donor may be from the same race or ethnic group as the subject. However, certain stem cells may be immune privileged and may be used allogeneically without matching between the donor and subject.

In one embodiment, the stem cell may be differentiated prior to use. In a non-limiting example, for tissue regeneration of cardiac tissue, it is possible to differentiate the stem cells into a cardiogenic precursor cells or cardiomyocytes prior to transplantation of the cells to the treatment site. In other embodiments, stem cells may be differentiated into kidney precursors, liver precursors, lung precursors, or other targeted tissue or organ precursor cell types.

The present method also can involve the co-administration of bioactive agents with the stem cells. By “co-administration” is meant administration before, concurrently with, e.g., in combination with bioactive agents in the same formulation or in separate formulations, or after administration of a therapeutic composition as described above.

As used herein, the phrase, “bioactive agents” refers to any organic, inorganic, or living agent that is biologically active or relevant. For example, a bioactive agent can be a protein, a polypeptide, a nucleic acid, a polysaccharide (e.g., heparin), an oligosaccharide, a mono- or disaccharide, an organic compound, an organometallic compound, or an inorganic compound. It can include a living or senescent cell, bacterium, virus, or part thereof. It may include a biologically active molecule such as a hormone, a growth factor, a growth factor producing virus, a growth factor inhibitor, a growth factor receptor, an anti-inflammatory agent, an antimetabolite, an integrin blocker, or a complete or partial functional sense or antisense gene, including siRNA. It can also include a man-made particle or material, which carries a biologically relevant or active material. An example is a nanoparticle comprising a core with a drug and a coating on the core.

Bioactive agents may also include drugs such as chemical or biological compounds that can have a therapeutic effect on a biological organism. Non-limiting examples include, but are not limited to, growth factors, anti-rejection agents, anti-inflammatory agents, anti-infective agents (e.g., antibiotics and antiviral agents), and analgesics and analgesic combinations. Anti-inflammatory agents may be useful as additional agents to counteract the inflammatory aspects of the fibrotic process.

Bioactive agents also can include precursor materials that exhibit the relevant biological activity after being metabolized, broken-down (e.g., cleaving molecular components), or otherwise processed and modified within the body. These may include such precursor materials that might otherwise be considered relatively biologically inert or otherwise not effective for a particular result related to the medical condition to be treated prior to such modification.

Combinations, blends, or other preparations of any of the foregoing examples may be made and still be considered bioactive agents within the intended meaning herein. Aspects of the present disclosure directed toward bioactive agents may include any or all of the foregoing examples.

In one embodiment, the bioactive agent may be a growth factor. A growth factor is any agent which promotes the proliferation, differentiation, and functionality of the implanted stem cell. Non-limiting examples of suitable growth factors may include, but are not limited to, leukemia inhibitory factor (LIF), epidermal growth factor (EGF), fibroblast growth factor (FGF), insulin-like growth factor (IGF), vascular endothelial growth factor (VEGF), human growth hormone (hGH), platelet-derived growth factor (PDGF), interleukins, cytokines, and/or combinations thereof.

In one embodiment, the bioactive agent may be an immunosuppressive agent. An immunosuppressive agent is an agent is any agent which prevents, delays the occurrence of, or decreases the intensity of the undesired immune response, e.g., rejection of a transplanted cell, tissue, or organ, or graft-versus-host disease. Preferred are immunosuppressive agents which suppress cell-mediated immune responses against cells identified by the immune system as non-self. Examples of immunosuppressive agents may include, but are not limited to, cyclosporin, cyclophosphamide, prednisone, dexamethasone, methotrexate, azathioprine, mycophenolate, thalidomide, FK-506, systemic steroids, as well as a broad range of antibodies, receptor agonists, receptor antagonists, and other such agents as known to one skilled in the art.

As used herein, “immunosuppression” refers to prevention of the immune response (for example by the administration of an “immunosuppressive agent”, as defined herein) such that an “immune response”, as defined herein, is not detectable. As used herein, “prevention” of an immune response means an immune response is not detectable. An immune response (for example, transplant rejection or antibody production) is detected according to methods well-known in the art and defined herein.

“Immunosuppression” as used herein also means a delay in the occurrence of the immune response as compared to any one of a transplant recipient that has not received an immunosuppressive agent, or a transplant recipient that has been transplanted with material that is not “immunologically blinded” or “immunoprivileged”, as defined herein. A delay in the occurrence of an immune response can be a short delay, for example 1 hour-10 days (i.e., 1 hour, 12 hours, 1 day, 2 days, 5 days, or 10 days. A delay in the occurrence of an immune response can also be a long delay, for example, 10 days-10 years (i.e., 30 days, 60 days, 90 days, 180 days, 1 year, 2 years, 5 years, or 10 years).

“Immunosuppression” as used herein also means a decrease in the intensity of an immune response. The intensity of an immune response can be decreased such that it is 5-100%, preferably, 25-100% and most preferably 75-100% less than the intensity of the immune response of any one of a transplant recipient that has not received an immunosuppressive agent, or a transplant recipient that has been transplanted with material that is not autologous. The intensity of an immune response can be measured by determining the time point at which transplanted material is rejected. For example, an immune response comprising rejection of transplanted material at day 1, post-transplantation, is of a greater intensity than an immune response comprising the rejection of transplanted material at day 30, post-transplantation. The intensity of an immune response can also be measured by quantitating the amount of a particular antibody capable of binding to the transplanted material, wherein the level of antibody production correlates directly with the intensity of the immune response. Alternatively, the intensity of an immune response can be measured by determining the time point at which a particular antibody capable of binding to the transplanted material is detected.

Various strategies and agents may be utilized for immunosuppression. For example, the proliferation and activity of lymphocytes can be inhibited generally with agents such as, for example, FK-506, or cyclosporin or other immunosuppressive agents. Another possible strategy may be to administer an antibody, such as an anti-GAD65 monoclonal antibody, or another compound which masks a surface antigen on a transplanted cell and therefore renders the cell practically invisible to the immune system of the host.

In other embodiments, bioactive agents that may be administered include anti-fibrotic agents including, but not limited to, nintedanib, INT-767, emricasan, VBY-376, PF-04634817, EXC 001, GM-CT-01, GCS-100, Refanalin, SAR156597, tralokinumab, pomalidomide, STX-100, CC-930, simtuzumab, anti-miR-21, PRM-151, BOT191, palomid 529, IMD1041, serelaxin, PEG-relaxin, ANG-4011, FT011, pirfenidone, F351 (perfenidone derivative), THR-184, CCX-140, FG-3019, avosentan, GKT137831, PF-00489791, pentoxifylline, fresolimumab, and LY2382770. In other embodiments, the bioactive agents may be Wnt/β-catenin signaling antagonists or TGF-β antagonists.

In another embodiment, the stem cells may be administered with a pharmaceutically acceptable carrier or excipients. The pharmaceutically acceptable excipients described herein, for example, vehicles, adjuvants, carriers, or diluents, are well-known to those who are skilled in the art and are readily available to the public. It is preferred that the pharmaceutically acceptable carrier or excipient be one which is chemically inert to the therapeutic composition and one which has no detrimental side effects or toxicity under the conditions of use.

The choice of excipient or carrier will be determined in part by the particular therapeutic composition, treated condition, as well as by the particular method used to administer the composition. Accordingly, there are a wide variety of suitable formulations of the pharmaceutical composition of the present invention. The formulations described herein are merely exemplary and are in no way limiting.

Often the physiologically acceptable carrier may be an aqueous pH buffered solution. Examples of physiologically acceptable carriers include, but are not limited to, saline, solvents, dispersion media, cell culture media, aqueous buffers such as phosphate, citrate, and other organic acids; antioxidants including ascorbic acid; low molecular weight (less than about 10 residues), polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The present disclosure further provides compositions useful in practicing the therapeutic methods described herein. A subject composition includes, in admixture, a pharmaceutically acceptable excipient (carrier) or media and the stem cells, including cells or tissues derived therefrom, alone or in combination with one or more bioactive agents, and at a strength effective for administration by various means to a patient suffering from tissue or organ fibrosis.

The preparation of cellular or tissue-based compositions is well understood in the art. Such compositions may be formulated in a pharmaceutically acceptable media. The cells may be in solution or embedded in a matrix. The preparation of compositions with bioactive agents (such as, for example, growth factors) as active ingredients is well understood in the art. The active therapeutic ingredient is often mixed with excipients or media which are pharmaceutically acceptable and compatible with the active ingredient. In addition, if desired, the composition can contain minor amounts of auxiliary substances such as wetting or emulsifying agents, pH buffering agents which enhance the effectiveness of the active ingredient.

A bioactive agent can be formulated into the composition as neutralized pharmaceutically acceptable salt forms. Pharmaceutically acceptable salts include the acid addition salts (formed with the free amino groups of the polypeptide or antibody molecule) which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In other embodiments, the compositions disclosed herein may be administered in a manner compatible with the dosage formulation, and in a therapeutically effective amount. The quantity to be administered depends, for instance, on the subject and debilitation to be treated, capacity of the subject's tissue, organ, cellular and immune system to accommodate the composition, and the nature of the cell or tissue therapy, etc. Precise amounts of composition required to be administered depend on the judgment of the practitioner and are peculiar to each individual. However, in some embodiments, suitable dosages of the composition disclosed herein may range from about 0.05-100.0×10⁶ stem cells per treatment site per day cells, or about 0.10-50.0×10⁶ stem cells per treatment site per day cells, or about 0.5-5.0×10⁶ stem cells per treatment site per day, and depend on the route of administration and the size of the treatment site. Suitable regimes for initial administration and follow on administration are also variable, but can include an initial administration followed by repeated doses at one or more hour, or day, intervals by a subsequent injection or other administration.

One of skill in the art may readily determine the appropriate concentration of cells for a particular purpose. In some embodiments, an exemplary dose is in the range of about 0.05-100.0×10⁶ cells per day. In a non-limiting example, approximately 0.5×10⁶ stem cells per treatment site per day, are injected adjacent to, or within, the treatment site (e.g., the tissue or organ).

In other embodiments, the stem cells described herein may be administered by injection into a target site of a subject, preferably via a delivery device, such as a tube, e.g., catheter. In one embodiment, the tube additionally contains a needle, e.g., a syringe, through which the stem cells can be introduced into the subject at a desired location. Specific, non-limiting examples of administering stem cells to subjects may also include administration by subcutaneous injection, intramuscular injection, or intravenous injection. If administration is intravenous, an injectable liquid suspension of stem cells can be prepared and administered by a continuous drip or as a bolus.

In further embodiments, stem cells may also be inserted into a delivery device, e.g., a syringe, in different forms. For example, the stem cells can be suspended in a solution contained in such a delivery device. As used herein, the term “solution” includes a pharmaceutically acceptable carrier or diluent in which the cells of the invention remain viable. The use of such carriers and diluents is well known in the art. The solution is preferably sterile and fluid to the extent that easy syringeability exists. In some embodiments, the solution is stable under the conditions of manufacture and storage and preserved against the contaminating action of microorganisms such as bacteria and fungi through the use of, for example, parabens, chlorobutanol, phenol, ascorbic acid, thimerosal, and the like. In other embodiments, solutions can be prepared by incorporating stem cells or differentiated cells as described herein, in a pharmaceutically acceptable carrier or diluent and, as required, other ingredients enumerated above, followed by filter sterilization.

In some embodiments, the cells may be administered systemically (for example intravenously) or locally (for example directly into a heart under echocardiogram guidance, or by direct application to an organ under visualization during surgery). For such injections, the stem cells may be in an injectable liquid suspension preparation or in a biocompatible medium which is injectable in liquid form and becomes semi-solid at the site of damaged tissue. A conventional intra-cardiac syringe or a controllable endoscopic delivery device can be used so long as the needle lumen or bore is of sufficient diameter (e.g., 30 gauge or larger) that shear forces will not damage the stem cells being delivered.

In other embodiments, stem cells may be administered in any manner that permits them to graft to the intended tissue site and reconstitute or regenerate the functionally deficient area.

In further embodiments, support matrices into which the stem cells can be incorporated or embedded include matrices which are biocompatible, recipient-compatible and which degrade into products which are not harmful to the recipient. These matrices provide support and protection for stem cells and differentiated cells in vivo.

Natural and/or synthetic biodegradable matrices are examples of such matrices. Natural biodegradable matrices may include, but are not limited to, plasma clots, e.g., derived from a mammal, collagen, fibronectin, and laminin matrices. Synthetic material for a cell transplantation matrix may be biocompatible to preclude migration and immunological complications, and should be able to support extensive cell growth and differentiated cell function and be resorbable, allowing for a completely natural tissue replacement. The matrix should be configurable into a variety of shapes and should have sufficient strength to prevent collapse upon implantation. Recent studies indicate that the biodegradable polyester polymers made of polyglycolic acid fulfill all of these criteria, as described by Vacanti et al. J. Ped. Surg. 23:3-9 (1988); Cima et al. Biotechnol. Bioeng. 38:145 (1991); Vacanti et al. Plast. Reconstr. Surg. 88:753-9 (1991). Other synthetic biodegradable support matrices include synthetic polymers such as polyanhydrides, polyorthoesters, and polylactic acid. Further examples of synthetic polymers and methods of incorporating or embedding cells into these matrices are also known in the art. See e.g., U.S. Pat. Nos. 4,298,002 and 5,308,701, which are both incorporated herein by reference for all they disclose regarding synthetic polymer matrices.

In some embodiments, attachment of the cells to the polymer may be enhanced by coating the polymers with compounds, which may include, but not limited to, basement membrane components, agar, agarose, gelatin, gum arabic, collagens types I, II, III, IV and V, fibronectin, laminin, glycosaminoglycans, mixtures thereof, and other materials known to those skilled in the art of cell culture. All polymers for use in the matrix must meet the mechanical and biochemical parameters necessary to provide adequate support for the cells with subsequent growth and proliferation.

One of the advantages of a biodegradable polymeric matrix is that angiogenic and other bioactive compounds can be incorporated directly into the support matrix so that they are slowly released as the support matrix degrades in vivo. As the cell-polymer structure is vascularized and the structure degrades, placental stem cells may differentiate according to their inherent characteristics. Factors, including nutrients, growth factors, inducers of differentiation or de-differentiation (i.e., causing differentiated cells to lose characteristics of differentiation and acquire characteristics such as proliferation and more general function), products of secretion, immunomodulators, inhibitors of inflammation, regression factors, bioactive agents which enhance or allow ingrowth of the lymphatic network or nerve fibers, hyaluronic acid, and drugs, which are known to those skilled in the art and commercially available with instructions as to what constitutes an effective amount, from suppliers such as Collaborative Research, Sigma Chemical Co., vascular growth factors such as vascular endothelial growth factor (VEGF), epidermal growth factor (EGF), and heparin binding epidermal growth factor like growth factor (HB-EGF), may be incorporated into the matrix or provided in conjunction with the matrix in some embodiments. Similarly, in other embodiments, polymers containing peptides such as the attachment peptide RGD (Arg-Gly-Asp) may be synthesized for use in forming matrices (see e.g., U.S. Pat. Nos. 4,988,621, 4,792,525, 5,965,997, 4,879,237 and 4,789,734, all of which are incorporated by reference herein for all they disclose regarding peptide-containing matrices).

In other embodiments, the cells may be transplanted in a gel matrix (such as Gelfoam® from Upjohn Company) which polymerizes to form a substrate in which the stem cells or differentiated cells can grow. in other embodiments, a variety of encapsulation technologies have been developed (e.g. Lacy et al., Science 254:1782-84 (1991); Sullivan et al., Science 252:718-712 (1991); International Patent Publication No. 91/10470; International Patent Publication No. 91/10425; U.S. Pat. Nos. 5,837,234; 5,011,472; 4,892,538, all of which are incorporated by reference herein for all they disclose regarding encapuslation technologies). In further embodiments, during open surgical procedures, involving direct physical access to the damaged tissue and/or organ, all of the described forms of undifferentiated stem cells or differentiated stem cell delivery preparations may be available options. These undifferentiated or differentiated stem cells may be repeatedly transplanted at intervals until a desired therapeutic effect is achieved.

### Examples

### Example 1: Administration of Energy Using an A1GaAs Low-Level Laser

An A1GaAs low-level laser comprising a wavelength of about 780 nm, a power output of about 3 mW, and a beam area of about 4 mm², is applied for about 30 seconds at a plurality of points on the surface of a kidney, for about 30 seconds at each point, with a delivered energy density of about 22.5 J/cm².

### Example 2: Administration of Energy Using a Low-Level Laser

An Intelect 800 laser (Chattanooga Corp., Chattanooga, Tenn.) comprising a wavelength of about 820 nm, an average power output of 50 mW, a beam spot size of about 1256 cm², and a power density of about 0.39 W/cm² is applied to an organ at 1 cm intervals around the periphery of the organ and across the surface in a grid of points placed at 1 cm intervals, with a delivered energy density of 20 J/cm² and a pulse repetition rate (PRR) of 5000 pulses per second (pps).

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A method for treating fibrosis of a tissue or organ in a subject in need thereof comprising the steps of:
(a) administering a therapeutically effective amount of energy from an energy source to the tissue or organ; and
(b) administering a therapeutically effective amount of stem cells to the tissue or organ,
wherein the energy reduces fibrosis in the tissue or organ, and wherein the stem cells repair or regenerate damaged portions of the tissue or organ.

2. (canceled)

3. A method for treating fibrosis of a tissue or organ in a subject in need thereof comprising the steps of:
(a) administering a therapeutically effective amount of energy from an energy source to the tissue or organ;
(b) detecting the level of fibrosis in the tissue or organ of the subject; and
(c) if fibrosis in the tissue or organ of the subject is reduced as a result of the energy application, administering a therapeutically effective amount of stem cells to the tissue or wherein the stem cells repair or regenerate damaged portions of the tissue or organ.

4. (canceled)

5. The method of claim 3, wherein the level of fibrosis is detected using an imaging method selected from computed tomography (CT), magnetic resonance imaging (MRI), ultrasound, and optical tomography.

6. The method of claim 3, wherein step (b) further comprises the steps of:
obtaining a biological sample from the subject;
(ii) measuring the amount of at least one marker of fibrosis in the biological sample;
(iii) comparing the amount of the at least one marker of fibrosis to a reference value.

7. The method of claim 6, wherein the reference value (i) is the amount of the at least one marker of fibrosis in a biological sample obtained from the subject prior to step (a), and wherein a reduction in the amount of the at least one marker of fibrosis as compared to the reference value indicates a reduction of fibrosis in the tissue or organ of the subject; or (ii) the amount of the at least one marker of fibrosis in a biological sample from a subject or subjects known to have fibrosis, and wherein a reduction in the amount of the at least one marker of fibrosis as compared to the reference value indicates a reduction of fibrosis in the tissue or organ of the subject.

8.-10. (canceled)

11. The method of claim 6, wherein the at least one marker of fibrosis is urea, creatinine, blood urea nitrogen, fibroblast-specific protein 1 (FSP-1), α-smooth muscle actin (α-SMA), interleukin 6 (IL-6), monocyte chemotactic protein-1 (MCP-1), transforming growth factor β1 (TGF-β1), or Smad3.

12.-16. (canceled)

17. The method of claim 1, wherein the energy source is a low-level laser, a pulsed laser, a calibrated laser, an adjustable laser, a continuous wave laser, or ultrasound.

18. (canceled)

19. (canceled)

20. The method of claim 17, wherein the energy source is a laser and the therapeutically effective amount of energy comprises a wavelength of about 780 nm, a power output of about 3 mW, and/or a beam area of about 4 mm², and wherein the energy is applied for about 30 seconds at each point of treatment.

21. The method of claim 17, wherein the energy source is a laser and the energy is delivered at a plurality of points on the surface of the tissue or organ, and wherein the energy is applied for about 30 seconds at each point.

22. The method of claim 17, wherein the delivered energy density is about 22.5 J/cm².

23. (canceled)

24. The method of claim 17, wherein the energy source is high-intensity focused ultrasound.

25. The method of claim 1, wherein the organ is selected from the group of kidney, liver, heart, lung, skin, intestine, and uterus.

26. (canceled)

27. The method of claim 1, wherein the stem cells are mesenchymal stem cells (MSC), adipose-derived stem cells (ADSC), adipose-derived side population stem cells (ADSC-SP), induced pluripotent stem cells (iPSC), or differentiated iPSC.

28.-33. (canceled)

34. The method claim 1, wherein the stem cells are autologous to the subject.

35. (canceled)

36. (canceled)

37. The method of claim 1, further comprising the step of administering to the subject a therapeutically effective amount of at least one agent for treating fibrosis.

38. The method of claim 35, wherein said agent is an anti-inflammatory agent or an anti-fibrotic agent.

39. The method of claim 1, further comprising the step of administering to the subject a therapeutically effective amount of at least one agent for preventing graft rejection.

40.-44. (canceled)

45. The method of claim 1, wherein the method treats a fibrotic condition.

46. The method of claim 43, wherein the fibrotic condition is thickening or scarring of connective tissue.

47. The method of claim 43, wherein the fibrotic condition is caused by injury, trauma, non-trauma, surgery, hereditary disease, or other chronic or non-chronic condition.

48. The method of claim 1, wherein the method treats a condition selected from the group consisting of adhesive capsulitis, arterial fibrosis, arthrofibrosis, Crohn's disease, cirrhosis, cystic fibrosis, Dupuytren's contracture, endomyocardial fibrosis, fibroleiomyoma, fibromyoma, idiopathic pulmonary fibrosis, keloid, mediastinal fibrosis, myelofibrosis, nephrogenic systemic fibrosis, old myocardial infarction, myoma, Peyronie's disease, progressive massive fibrosis, pulmonary fibrosis, retroperitoneal fibrosis, scleroderma/systemic sclerosis, uterine fibroids, uterine leiomyoma, and other conditions relating to excessive connective tissue.
